(19) United States
(12) Patent Application Publication
(10) Pub. No.: US 2022/0015739 A1
(43) Pub. Date: Jan. 20, 2022

(54) RESPIRATORY VARIATION IN INTERNAL JUGULAR VEIN DIAMETER AS A METHOD FOR ESTIMATING PATIENT'S VOLUME STATUS AND VENTRICULAR FUNCTION

(71) Applicant: Gaurang Nandkishor Vaidya, Lexington, KY (US)

(72) Inventor: Gaurang Nandkishor Vaidya, Lexington, KY (US)

(21) Appl. No.: 17/486,913

(22) Filed: Sep. 28, 2021

(51) Int. Cl. A61B 8/08; A61B 8/00; A61B 8/04

(52) U.S. Cl. CPC A61B 8/0883; A61B 8/5207; A61B 8/42; A61B 8/04; A61B 8/461; A61B 8/543

(57) ABSTRACT

The present invention comprises a novel method to utilize the diameter of the internal jugular vein, obtained using an ultrasound machine, to estimate a patient's volume status and cardiac ventricular function. In this technique, the ultrasound machine is used to measure the diameter of the internal jugular vein (IJV) lumen. In addition, the ultrasound machine is used to measure the respiratory variation in the IJV lumen diameter, measured as the difference between the maximum and minimum diameter divided by the maximum diameter and expressed as a percentage. The ultrasound machine is also used to identify a complete approximation of the IJV diameter into 0 millimeters with deep breathing and/or sniff. The above information is used to estimate the patient's volume and cardiac function status.

### FIELD OF INVENTION

The present invention is related to the use of ultrasound measurement of internal jugular vein diameter and utilization of this information to ascertain a person's volume status and cardiac function. More particularly, the invention relates to the use of portable imaging technology for vital bedside assessment of patients and assist in daily patient care decisions.

### BACKGROUND

Assessment of a patient's volume status is a common daily assignment for clinicians. An accurate estimation of a patient's body water, especially an excess within the vascular system, is of paramount importance in the management of heart failure patients. In addition, this is also necessary in multiple other conditions including kidney failure, septic shock, pulmonary hypertension, and endocrine disorders.

The current gold standard for an accurate estimation of volume status is a right heart catheterization, which involves inserting a catheter into the various chambers of the heart and lungs to record the pressures directly. This however is an invasive and expensive procedure, only performed by credentialed physicians and not feasible for daily bedside use. The non-invasive alternative is a physical examination of the neck to ascertain the level of pulsation of the internal jugular vein, the use of which can be confounded by a patient's body habitus and occasionally unobtainable in obese individuals. Ultrasound of the inferior vena cava can be employed for this purpose; however, this is a technique requiring considerable training, unobtainable in obese patients and result in patient discomfort from the need to expose the patient's abdomen and exert manual pressure on the midriff.

Similarly, an assessment of patient's cardiac function is currently performed by trained sonographers and cardiologists using an echocardiogram. To obtain accurate and useful heart ultrasound images, a physician must commit multiple months towards dedicated training.

Therefore, a need exists to identify a non-invasive modality which could provide objective and accurate assessment of a patient's volume and cardiac status, which could also be useful on a daily basis, be portable for use at bedside, be easy to learn for all physicians at various levels of training and be less discomforting to the patient.

### BRIEF SUMMARY OF THE EMBODIMENT

The present invention comprises a novel method to utilize the diameter of the internal jugular vein, obtained using an ultrasound machine, to estimate a patient's volume status and cardiac ventricular function. In this technique, the ultrasound machine is used to measure the diameter of the internal jugular vein (IJV) lumen. In addition, the ultrasound machine is used to measure the respiratory variation in the UV lumen diameter, measured as the difference between the maximum and minimum diameter divided by the maximum diameter and expressed as a percentage. The ultrasound machine is also used to identify a complete approximation of the IJV diameter into 0 millimeters with deep breathing and/or sniff. The above information is used to estimate the patient's volume and cardiac function status.

### BRIEF DESCRIPTION OF THE DRAWINGS

Some embodiments of the present invention are illustrated as an example and are not limited by the figures of the accompanying drawings, in which like references may indicate similar elements and in which:

FIG. 1: FIG. 1 illustrates a non-limiting example of the method of patient positioning to expose the internal jugular vein (marked as #2) and positioning of the ultrasound probe (marked #1) at the apex of the sternocleidomastoid muscle (marked #3)

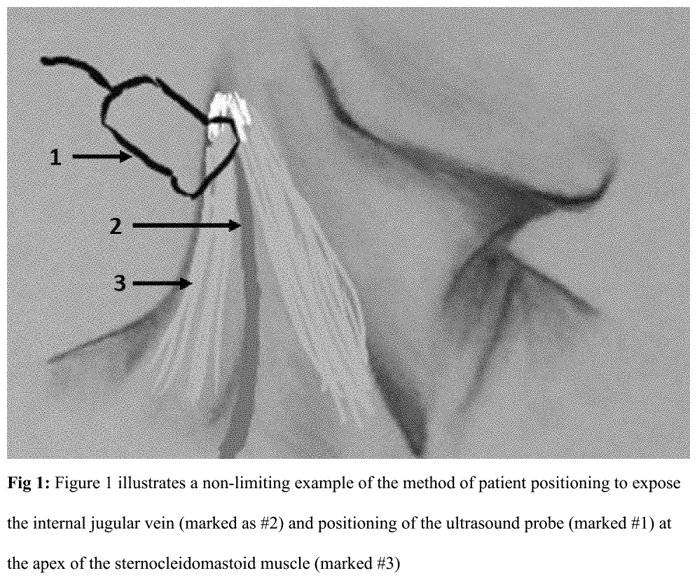

FIG. 2: FIG. 2 illustrates a non-limiting example of the method to measure the diameter of the internal jugular vein-using M-mode of the ultrasound. Maximum (marked #1) and minimum (marked #2) diameter are identified over a time spanning one respiratory cycle.

FIG. 3: FIG. 3 illustrates a non-limiting example of the method to estimate subject's fluid status and intracardiac pressure using an algorithm derived from the non-limiting study quoted in the detailed description of the embodiment

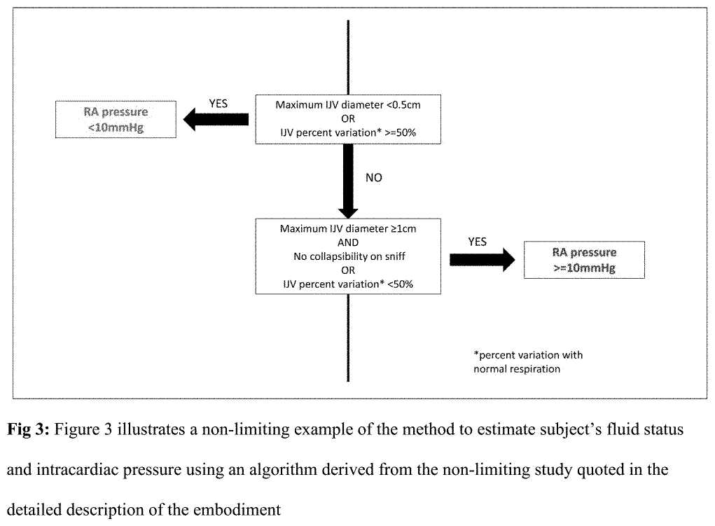

FIG. 4: FIG. 4 illustrates a non-limiting example of a computing device capable of computing internal jugular vein dimensions to display estimated intra-cardiac pressure

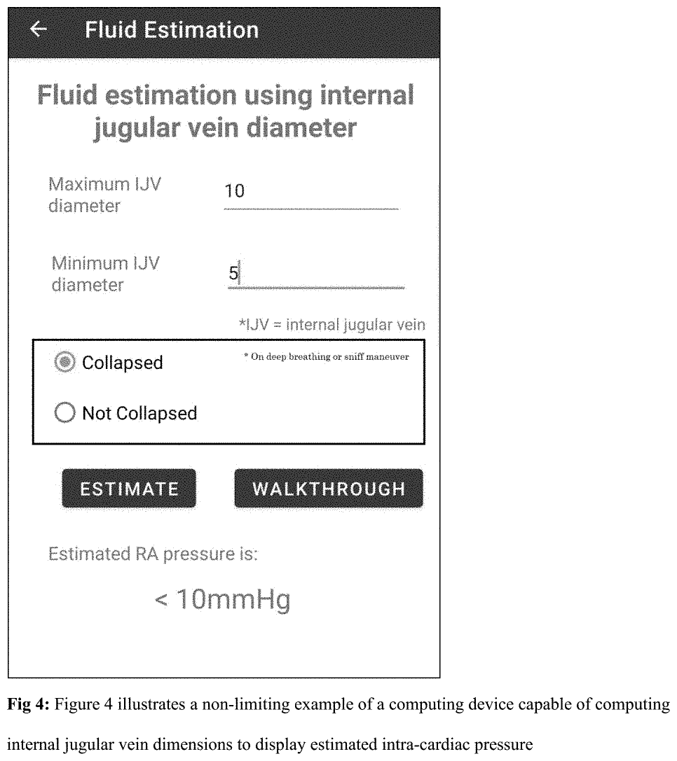

### DETAILED DESCRIPTION OF THE EMBODIMENT

In the following detailed description, references are made to the accompanying drawings in which are shown the illustrations in how the embodiments may be practiced. It is to be understood that other embodiments may be utilized, with or without structural, procedural or logical changes, without departing from the scope. Therefore, the following description is not to be taken in a restricted or all-inclusive sense, and the scope of embodiments is defined by the appended claims and their equivalents. Moreover, the order of description of various procedures below should not be construed to imply that the procedures are order-dependent.

The description may use perspective-based descriptions such as up/down, back/front, supine/erect, anterior/posterior and top/bottom. Such descriptions are merely used to facilitate the discussion and are not intended to restrict the application of disclosed embodiments.

For the purposes of the description, a phrase in the form “A/B” or in the form “A and/or B” means (A), (B), or (A and B). For the purposes of the description, a phrase in the form “at least one of A and B” means (A), (B) or (A and B). The description may use the terms “embodiment” or “embodiments,” which may each refer to one or more of the same or different embodiments.

In the description, various apparatuses are described for use to carry out the various methods within the claims. In the future, a device may be endowed with the imaging and computing ability to perform all or various individual functions together and such a device may be employed to carry out the various methods as described below.

Embodiments described below provide methods to assess a subject's volume status, intra-cardiac pressures and cardiac function using an ultrasound of the internal jugular vein. These ultrasound assessments have been correlated with direct intra-cardiac pressure measurement and cardiac function evaluation through gold-standard right heart catheterization technique.

The methods involve acquisition of ultrasound imaging data of the right or left internal jugular vein (IJV) in the subject; utilization of the ultrasound image data for measurement of IJV dimensions; processing the IJV dimension data and determining the volume status and intra-cardiac pressure from the IJV dimension data. The data is thereafter displayed on a display device and stored for future use. The embodiment also describes acquisition of respiratory data concerning the IJV; and determination of intra-cardiac pressure utilizing both IJV dimensions and respiratory data. Intra-cardiac pressure includes both right and left intra-cardiac pressures.

According to some embodiment, the measurement of IJV dimension data comprises the maximum and minimum diameter of the IJV lumen. In some embodiment, processing the IJV dimension data comprises calculating the difference between the maximum and minimum diameter of the IJV lumen and/or percentage variation in the IJV diameter over a time span, for instance the time span of one full inspiration and expiration cycle of respiration. In other embodiments, the methods include the processing the IJV dimension data which further comprises of determining the extent of IJV diameter diminution on deep/full inspiration and/or sniff maneuver by the subject.

The description below also provides a method to assess cardiac function through an acquisition of ultrasound imaging data of the internal jugular vein (IJV) in the subject; utilization of the ultrasound image data for measurement of IJV diameter variation over a time span; processing the IJV diameter data and determining the indicators of cardiac function from the IJV diameter data. The time span comprises for instance at least one respiratory cycle including an inspiration and an expiration. The indicators of cardiac function include an estimate of the right ventricular stroke work index and/or pulmonary artery pulsatility index. The estimated cardiac function is displayed on a display device and stored for future use.

The techniques may be implemented according to various embodiments described herein using an ultrasound device capable of imaging the right or left internal jugular vein and have an ability to measure the diameter, for instance using M-mode technique within the ultrasound. The ultrasound device may be capable of trending the diameter variation as occurring during the respiratory cycle of a subject, for instance using M-mode technique within the ultrasound. According to embodiments, the images may then be displayed on a display monitor. The acquired IJV dimension and respiratory data is provided to a image processing or computing device for determination of volume status, intra-cardiac pressures and/or cardiac function.

Assessment of a patient's volume status is a common daily assignment for clinicians. An accurate estimation of a patient's body water, especially an excess within the vascular system, is of paramount importance in the management of heart failure patients. Moreover, the utility of this assessment spans beyond cardiologists to also include internists, hospitalists, family medicine practitioners, nephrologists etc. Currently the only techniques feasible for daily bedside use and also widely practiced to provide a volume estimation is the physical examination of the jugular venous distension which can be inaccurate and unreliable. Imaging the inferior vena cava is feasible at bedside but this comprises a significant learning curve for most physicians and a potential for discomfort to the patients related to being exposed and manually pushed on at the midriff. The gold standard for volume estimation is a right heart catheterization (RHC) which is not feasible for daily use, invasive and expensive.

On the other hand, ultrasound of the internal jugular vein (IJV) is a comparatively easier skill to acquire given the superficial nature of the vein. Bedside ultrasound is possible using portable ultrasound machines widely available in all hospitals. Portable ultrasound probes are also available for commercial purchase by individual practitioners, which have the ability to connect with most smartphones and have processing and computing ability embedded within the probe. A portable ultrasound of the IJV has the ability to provide a truly objective and accurate bedside assessment of a subject's fluid status and cardiac function, with minimal learning curve for practitioners and with minimal to no discomfort to the patients. This technique could be vital, for instance, in the hands of primary care practitioners within community centers and outreach hospitals.

Thus, enclosed herein, in various embodiments are methods to utilize ultrasound assessment of IJV to make an accurate estimation of the subject's volume status and cardiac function. The accuracy of the obtained IJV dimension and respiratory data was demonstrated on correlation with simultaneously performed RHC.

In a specific, non-limiting embodiment, 72 patients scheduled to undergo RHC within the Jewish hospital and University of Louisville hospital in Louisville were enrolled in a prospective study. The study protocol was reviewed and approved by the institutional review board of the University of Louisville. All patients included in the study signed an informed consent. The inclusion criteria included: spontaneously breathing adults (age >18 years) and able to consent. Patients with orthotopic heart transplant (OHT) or left ventricular assist device (LVAD) were also eligible for enrollment. Exclusion criteria included: known occlusion of IJV, superior vena cava obstruction/compression or severe tricuspid regurgitation.

For the purpose of the study, patients were educated about the study procedures including the sniff maneuver. For standardization of patient positioning within this specific and non-limiting embodiment, patients were then positioned supine at 0 degrees with their head in neutral position and breathing restfully. Next, the right sternocleidomastoid muscle was identified and the right IJV was imaged at the apex of the triangle formed by the sternal and clavicular heads of the muscle. If the patient had an indwelling intravenous catheter or an implanted device, such as a pacemaker, on one side of the neck or chest wall then the left IJV was used.

For this particular non-limiting embodiment, a portable ultrasound system-Sonosite (Bothell, Wash.) was used for imaging purposes. Using M-mode technique of the ultrasound, the maximum and minimum anteroposterior diameters of IJV at the above-mentioned landmarks, were noted during normal breathing, without applying any external pressure.

The respiratory variation in diameter (RVD) was calculated as [(maximum diameter−minimum diameter)/maximum diameter] and expressed as percent. The patients were then asked to sniff forcefully. The anteroposterior diameter collapsibility was assessed on sniff maneuver. The first 10 imaging acquisitions were timed.

The patients then underwent right heart catheterization within 1 h of the ultrasound assessment and right atrial (RA) pressure, right ventricular (RV) pressure, pulmonary artery (PA) pressure and pulmonary capillary wedge pressure (PCWP) were recorded.

IBM SPSS (version 24.0, SPSS Corp, Chicago, Ill., USA) was used for statistical analysis. Qualitative data is presented as frequencies and quantitative data as mean±standard deviation. Categorical variables and continuous variables were analyzed using Chi-square test, and Student's t-test respectively. The correlation of imaging parameters to invasive RA pressure measurement was assessed using linear regression. Receiver operating curve (ROC) analysis was performed to determine the sensitivity and specificity of imaging parameters in estimation of right atrial pressure with the invasive RA pressure as the gold standard. A two-sided p-value <0.05 was considered significant.

Total of 72 patients were enrolled in the study with mean age 61±14 years, and mean BSA 1.9±0.2 m2. None of the patients were ventilator dependent or on intravenous inotropic/vasoactive agents. Echocardiography data was available in 81% of patients within one month of enrollment and the mean LVEF was 45% (10-75%). Forty percent of patients had BMI ≥30 kg/m2 (table 1A).

TABLE 1A
Baseline characteristics of the patient population

| Variable | Frequency/Mean |
|---|---|
| Male | 61% |
| Age (years) | 60.8 ± 14.0 (21-85) |
| Body surface area (m²) | 1.9 ± 0.2 (1.3-17.4) |
| Body mass index (kg/m²) | 30.0 ± 6.5 (17.4-48.1) |
| Systolic blood pressure (mmHg) | 125.4 ± 24.0 (54-196) |
| Heart rate (beats/min) | 75.8 ± 15.5 (52-110) |
| Atrial fibrillation | 9% |
| Trace/mild tricuspid regurgitation | 89% |
| LV ejection fraction (%) | 45.2 ± 20.0 (10-75) |
| Recurrent catheterization | 14% |
| Serum Creatinine (mg/dL) | 1.47 ± 1.46 (0.39-10.56) |
| Blood urea nitrogen/creatinine ratio | 17.31 ± 6.6 (4.0-37.2) |
| Serum bicarbonate (mg/dL) | 25.8 ± 3.5 (11.3-34.0) |

Normal LVEF was defined as ≥52% in males and ≥54% in females based on American Society of Echocardiography guidelines. Half the patients with available data had normal ejection fraction and 42% had EF≤35%. The cohort included 6 (8%) OHT recipients and 4 (6%) patient with LVAD implantation.

Image acquisition required <5 minutes per patient as assessed in the first 10 patients. IJV could be imaged in all patients irrespective of their body mass index (BMI).

Right heart catheterization findings are described in table 1B; 35% of patients had RA pressure ≥10 mmHg and 31% had at least moderate pulmonary hypertension. All patients with maximum IJV diameter <0.5 cm had RA pressure <10 mmHg (12 patients, 17%). Similar findings were noted with RVD in IJV >50% (16 patients, 22%).

TABLE 1B
Right heart catheterization findings

| Variable (mmHg) | Mean |
|---|---|
| Right atrial pressure (mmHg) | 8.3 ± 5.3 (0-20) |
| Pulmonary systolic pressure (mmHg) | 44.7 ± 20.2 (16-120) |
| Pulmonary mean pressure (mmHg) | 28.8 ± 13.0 (10-74) |
| Pulmonary capillary wedge pressure (mmHg) | 15.5 ± 9.3 (4-48) |

Patients with elevated RA pressure (≥10 mmHg) showed less RVD in IJV with respiration in resting condition (14 vs. 40%, p=0.01). They also had larger maximum IJV diameter (p=0.01) [table 2].

TABLE 2
Elevated RA pressure and IJV diameter variation on respiration

| | RA pressure | RA pressure | |
|---|---|---|---|
| | ≥10 mmHg | <10 mmHg | P-value |
| Maximum IJV | 1.0 ± 0.2 | 0.7 ± 0.3 | 0.001 |
| diameter (cm) | | | |
| Percent IJV diameter | 14% | 40% | 0.001 |
| variation | | | |
| Complete IJV AP | 16% | 66% | 0.001 |
| collapsibility on sniff | | | |

Complete collapsibility of IJV anteroposterior diameter with sniff maneuver was associated with significantly lower RA (5.2 vs 11.3 mmHg, p=0.001) and PCWP pressures (12.2 vs 18.5 mmHg, p=0.004) [table 3].

TABLE 3
IJV collapsibility on sniff and the right heart catheterization findings

| | IJV collapsible | Not collapsible | P-value |
|---|---|---|---|
| Right atrial pressure | 5.2 ± 2.8 | 11.3 ± 5.4 | 0.001 |
| (mmHg) | | | |
| Pulmonary systolic | 36.2 ± 12.7 | 52.7 ± 22.7 | 0.001 |
| pressure (mmHg) | | | |
| Pulmonary mean | 23.2 ± 8.2 | 34.1 ± 14.4 | 0.001 |
| pressure (mmHg) | | | |
| Pulmonary capillary | 12.2 ± 7.3 | 18.5 ± 10.1 | 0.004 |
| wedge pressure | | | |
| (mmHg) | | | |

Sensitivity and specificity analysis were performed to assess accuracy of IJV ultrasound in estimating high RA pressure (table 4). For RA pressure ≥10 mmHg, lack of IJV collapsibility with sniff had a sensitivity of 84% and specificity of 66%. A maximum IJV diameter ≥1 cm and respiratory variation <50% had a sensitivity of 60% and specificity of 80% with ROC area 0.694 for RA pressure ≥10 mmHg (table 4). Similarly, a maximum IJV diameter ≥1 cm and lack of complete IJV collapsibility with sniff had a sensitivity and specificity of 56% and 83% respectively.

TABLE 4
Sensitivity and specificity of various IJV findings in predicting
RA pressure ≥10 mmHg.

| | | | ROC |
|---|---|---|---|
| | Sensitivity | Specificity | AUC |
| Maximum IJV diameter ≥1 cm | 60% | 72% | 0.662 |
| No IJV collapsibility with sniff | 84% | 66% | 0.750 |
| Maximum IJV diameter ≥1 cm + | 56% | 83% | 0.708 |
| no collapsibility | | | |
| Maximum IJV diameter ≥1 cm + | 60% | 80% | 0.694 |
| percent variation <50% on | | | |
| normal respiration | | | |

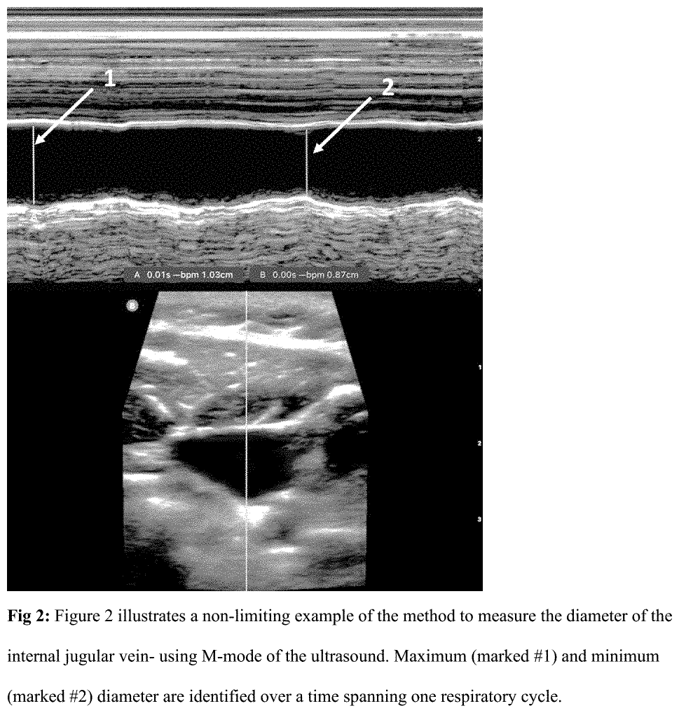

Among the subgroup of patients with EF≤35% (30 patients), the percent diameter variation continued to have positive correlation with RA pressures (R=0.66, p=0.001). Similarly, for patients with mean pulmonary pressure ≥35 mmHg, the positive correlation between percent variation and RA pressure was maintained (R=0.66 for IJV, p=0.001). Based on the above data, an algorithm was constructed to estimate RA pressure at bedside with considerable certainty (FIG. 2). There was a positive correlation between IJV diameter difference and pulmonary artery pulsatility index (PAPi) (p=0.012).

The study reports, a strong positive correlation of internal jugular vein diameters as well as their collapsibility, as assessed with bedside ultrasound, with invasive right heart catheterization. The study cohort represents a real-world population of patients including patients with heart failure, pulmonary hypertension, LVAD and heart transplant. The study demonstrated 1) A significant positive correlation between the vein diameters and RA pressure, 2) Less respiratory variation and larger vein diameters with elevated RA pressure, and 3) Lack of IJV collapsibility as a highly sensitive marker for higher RA pressure. The study results have direct application in day-to-day care of patients.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein.

What is claimed:

1. A method for assessing a subject's volume status and intra-cardiac pressures comprising:
acquisition of ultrasound imaging data of the internal jugular vein (IJV) in the subject; utilization of the ultrasound image data for measurement of IJV dimensions; processing the IJV dimension data and determining the volume status and/or intra-cardiac pressure from the IJV dimension data.

2. The method of claim 1, further comprising: acquisition of respiratory data concerning the IJV; and determination of intra-cardiac pressure utilizing both IJV dimensions and respiratory data

3. The method of claim 1, further comprising displaying the estimated volume status and/or intra-cardiac pressure on a display device

4. The method of claim 1, further comprising of recording the volume status

5. The method of claim 1, wherein the measurement of IJV dimension data comprises the maximum and minimum diameter of the IJV lumen.

6. The method of claim 5, wherein processing the IJV dimension data comprises calculating the difference between the maximum and minimum diameter of the IJV lumen and/or percentage variation in the IJV diameter over a time span

7. The method of claim 6, wherein the time span comprises of one full inspiration and expiration cycle of respiration

8. The method of claim 6, wherein percentage variation in the IJV diameter comprises the calculation of the difference between the maximum and minimum IJV diameter divided by the maximum IJV diameter

9. The method of claim 7, wherein the processing the IJV dimension data further comprises of determining the extent of IJV diameter diminution on deep/full inspiration and/or sniff maneuver by the subject.

11. A method to evaluate cardiac function of a subject, the method comprising: acquisition of ultrasound imaging data of the internal jugular vein (IJV) in the subject; utilization of the ultrasound image data for measurement of IJV diameter variation over a time span; processing the IJV diameter data and determining the indicators of cardiac function from the IJV diameter data.

12. The method of claim 11, wherein the time span is at least one respiratory cycle comprising an inspiration and an expiration.

13. The method of claim 11, wherein the indicator of cardiac function is an estimate of the right ventricular stroke work index and/or pulmonary artery pulsatility index

14. The method of claim 11, further comprising displaying the estimated cardiac function on a device

15. The method of claim 11, further comprising of recording the cardiac function.
